(19) United States
(12) Patent Application Publication
(10) Pub. No.: US 2025/0249290 A1
(43) Pub. Date: Aug. 7, 2025

(54) METHODS AND SYSTEMS OF IMPROVING TINNITUS VIA ULTRASOUND NEUROMODULATION OF THE BRAIN

(71) Applicant: West Virginia University Board of Governors on behalf of West Virginia University, Morgantown, WV (US)

(72) Inventors: Ali R. Rezai, Morgantown, WV (US); Victor Finomore, Morgantown, WV (US); Mark Schafer, Morgantown, WV (US)

(21) Appl. No.: 19/046,805

(22) Filed: Feb. 6, 2025

Related U.S. Application Data
(60) Provisional application No. 63/550,066, filed on Feb. 6, 2024.

(51) Int. Cl. A61N 7/00

(52) U.S. Cl. CPC A61N 7/00; A61N 2007/0021; A61N 2007/0086

(57) ABSTRACT

Methods and systems of improving tinnitus in a patient in need thereof is provided. The method can include delivering an ultrasound signal to a target site of the patient's brain. The target site can include the medial geniculate nucleus, the nucleus accumbens, the caudate nucleus, the pulvinar nucleus, the insula, the subcallosal anterior cingulate area, the cingulate cortex, the auditory cortex, or combinations thereof. The method can be performed without delivering microbubbles to open the blood brain barrier. The ultrasound signal can have a Mechanical Index of between about 1.5 and about 5.0 and/or an acoustic pressure of between about 0.55 MPa and about 2.5 MPa.

### CROSS-REFERENCE TO RELATED APPLICATIONS

The present application claims priority to U.S. Provisional Application No. 63/550,066 filed on Feb. 6, 2024, which is incorporated by reference in its entirety.

### TECHNICAL FIELD

The present technology is directed to systems and methods for improving tinnitus by delivering a focused ultrasound signal (FUS) to a neural target site of the brain.

### BACKGROUND

Tinnitus is a complex disorder that is the result of conscious perception of a tone or composite noise that has no corresponding external auditory source. Tinnitus disorder can include cognitive impairment, emotional discomfort and functional disability. Tinnitus is a frequent auditory disorder and approximately 10% of patients suffering from tinnitus develop mild or severe impairment. Severe tinnitus is strongly linked to anxiety, sleeplessness, difficulties concentrating, poor psychological well-being, and poor quality of life.

FDA-approved drugs indicate that none of the drugs tested completely abolished tinnitus. In addition to drugs, an increasing number of both invasive and non-invasive techniques, such as cochlear implants, removal of the vestibulo-cochlear nerve, surgical decompression of the auditory nerve from the compressed veins, transcranial magnetic stimulation (TMS), transcranial electrical stimulation (TES), and other techniques have been used in the treatment of tinnitus. However, there is insufficient evidence supporting the effectiveness of these interventions.

### SUMMARY

In an aspect, a method of improving tinnitus in a patient in need thereof is provided. The method can comprise delivering a focused ultrasound neuromodulation signal to a neural target site of the patient's brain. The ultrasound signal can have a Mechanical Index of between about 1.5 to about 5.0 and/or an acoustic pressure of between about 0.55 MPa to about 2.5 MPa. The target site can comprise a medial geniculate nucleus, a nucleus accumbens, a caudate nucleus, a pulvinar nucleus, an insula, a subcallosal anterior cingulate area, a cingulate cortex, an auditory cortex or combinations thereof. The method can be performed without delivering microbubbles to open the blood brain barrier.

In another aspect, a system of improving tinnitus is provided. The system can comprise an ultrasound transducer, a processor, and a non-transitory computer-accessible medium having stored thereon computer-executable instructions which, when executed by a processor, directs an ultrasound transducer to deliver an ultrasound signal to a target site of the patient's brain to improve tinnitus. The ultrasound signal can comprise a Mechanical Index of between about 1.5 and about 5.0 and/or an acoustic pressure of between about 0.55 MPa and about 2.5 MPa.

In another aspect, a non-transitory computer-accessible medium is provided. The computer-accessible medium has stored thereon computer-executable instructions which, when executed by a processor, directs an ultrasound transducer to deliver an ultrasound signal to a target site of the patient's brain to improve tinnitus, the ultrasound signal comprising a Mechanical Index of between about 1.5 and about 5.0 and/or an acoustic pressure of between about 0.55 MPa and about 2.5 MPa.

### BRIEF DESCRIPTION OF THE DRAWINGS

FIG. 1 is a flow diagram outlining steps of a method of improving tinnitus according to an aspect of the present disclosure.

FIG. 2 is a block diagram depicting illustrative components of a system to improve tinnitus according to an aspect of the present disclosure.

FIG. 3 is a block diagram depicting illustrative components of a system to improve tinnitus according to an aspect of the present disclosure.

### DETAILED DESCRIPTION

As used herein with respect to a described element, the terms “a,” “an,” and “the” include at least one or more of the described element(s) including combinations thereof unless otherwise indicated. Further, the term “or” includes “and” and combinations thereof unless otherwise indicated. The term “and” refers to combinations thereof unless otherwise indicated. By “substantially,” “approximately, or “about” is meant that the value of the described element need not have the mathematically exact described value of the described element but can have a value that is recognizable by one skilled in the art as generally or approximately having the described value of the described element. As such “substantially,” “approximately,” or “about” refers to the complete or nearly complete extent of a value. The exact allowable degree of deviation from the value will be so as to have the same overall result as if the absolute characteristic, property, state, structure, or value were obtained. A “patient” as used herein is a mammal such as, for example, a human being, dog, cat, horse, pig, sheep, cow, or other domesticated animal.

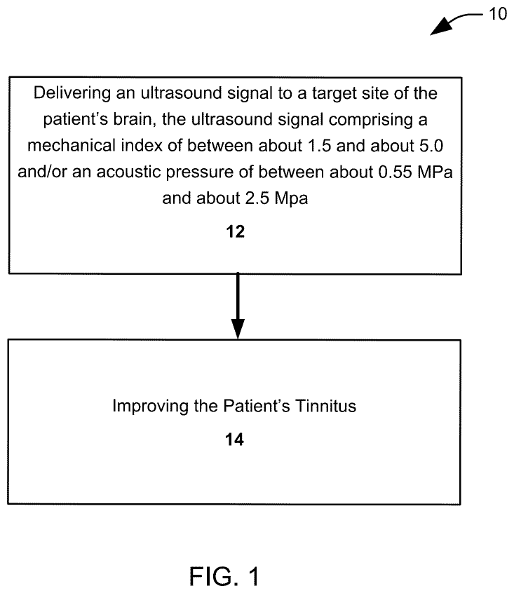

In an aspect and with reference to FIG. 1, a method of improving tinnitus 10 in a patient in need thereof is provided. The method can include delivering an ultrasound signal to a target site of the patient's brain 12 comprising a medial geniculate nucleus, a nucleus accumbens, a caudate nucleus, a pulvinar nucleus, an insula, a subcallosal anterior cingulate area, a cingulate cortex, an auditory cortex, or combinations thereof. The method can be performed without delivering microbubbles to open the blood brain barrier (BBB). In other words, in certain aspects, no microbubbles are injected into the blood after which the brain is treated with focused ultrasound. As such, the ultrasound will not cause such microbubbles to vibrate, which would create mechanical forces that open the BBB. The ultrasound signal can have a Mechanical Index of between about 1.5 and about 5.0 and/or an acoustic pressure of between about 0.55 MPa and about 2.5 MPa. The focused ultrasound neuromodulation signal can be non-ablative.

Focused ultrasound can be defined as an acoustic wave above 20 kHz, beyond the frequency range of human hearing. An ultrasound transducer, as used herein, is a device that produces an ultrasound signal, or beam. Transducers can be implemented in arrays to allow for electrical steering of the device. An ultrasound transducer and ultrasound device are used interchangeably herein.

Table I provides examples of FUS/sonication parameters that can be measured to improve tinnitus.

TABLE I
Sonication parameters.

| | Abbrev. | |
|---|---|---|
| Parameter | (Unit) | Description |
| Ultrasound | f₀ (kHz or | The frequency of ultrasonic pressure |
| operating | MHz) | waves; the carrier frequency of the |
| frequency | | ultrasound signal |
| Pulse duration | PD (ms) | The shortest continuous period of |
| | | sonication. When applying continuous |
| | | TUS, pulse duration is equal to the |
| | | pulse train duration; the duration of a |
| | | pulse in a series of ultrasound pulses |
| Pulse repetition | PRF (Hz) | The frequency of pulse delivery within |
| frequency/rate | | the pulse train duration; multiplicative |
| | | inverse of the pulse repetition interval |
| Duty cycle | DC (%) | The percentage of time sonication is |
| | | delivered during the pulse repetition |
| | | interval. The duty cycle is only defined |
| | | for rectangular pulses. (or pulse train |
| | | duration) |
| Spatial | pₛₚ | The position at which the ultrasound |
| peak/spatial peak | | signal has a highest amplitude. The |
| pressure | | spatial peak occurs within a focal spot |
| | | of the ultrasound transducer; the |
| | | amplitude of the pressure during the |
| | | steady part of the pulse at the location |
| | | of the spatial peak. |
| Spatial peak pulse | I_{SPPA} | The intensity of the pulse at the spatial |
| average intensity | (W/cm²) | peak (focal peak) time averaged over |
| | | the pulse duration |
| Spatial peak | I_{SPTA} | The intensity of the ultrasonic stimulus |
| temporal average | (mW/cm²) | at the spatial peak time averaged over |
| intensity | | a defined period of time (i.e., across the |
| | | stimulus duration). The temporal |
| | | window must be defined. |
| Mechanical Index | MI | A unitless index for assessing the |
| | | likelihood of cavitational bioeffects. |
| | | Where p_{r.3} is the pressure derated using |
| | | αα = 0.3 dB cm⁻¹ MHz⁻¹ |
| Acoustic Pressure | Megapascal | Amount of force applied per unit area |
| | (MPa) | and is typically thought of as a local |
| | | pressure deviation relative to the |
| | | ambient surround pressure |
| Power | Watts | The power of the electrical waveform |
| | | emitted by ultrasound pulse generator |
| | | which is amplified and carried to one or |
| | | more ultrasound transducers |

Z = Acoustic Impedance

In further detail, to characterize the intensity of a pulsing protocol, a needle hydrophone can be used to measure the instantaneous pressure (Pᵢ) in Megapascals applied by the transducer in an acoustic medium. The instantaneous intensity (Iᵢ) is proportional to the square of the instantaneous pressure, and inversely related to the density (ρ) and speed of sound (c) in the propagating medium. The pulse intensity integral (PII) is then derived by integrating the instantaneous intensity over the duration of the pulse. From the PII, two measures of acoustic exposure can be derived: the spatial-peak temporal average (I_{SPTA}) and the spatial-peak pulse average (I_{SPPA}). I_{SPTA} measures the average intensity during the entire sonication and scales in proportion to sonication duration. Conversely, I_{SPPA} represents the average intensity over a single pulse, providing an estimate of short-term mechanical bioeffects. Another parameter is acoustic pressure, which is the amount of force applied per unit area and is typically thought of as a local pressure deviation relative to the ambient surround pressure. It is commonly represented with the symbol p and is given in Megapascal (MPa) units. Another parameter that can be measured is the Mechanical Index (MI), a unitless measure which is equal to peak negative pressure divided by the square of the fundamental frequency. The MI can estimate the risk of potentially destructive biomechanical effects on tissues, such as inertial cavitation. Frequency can also be measured, which is the number of wave cycles per second and is determined by the rate at which the ultrasound source oscillates and influences many aspects of tissue interaction. It is commonly represented with the symbol f and is given in Megahertz (MHz) units.

The focused ultrasound signal can be generated with suitable stimulation parameters. In one implementation, focused ultrasound is provided with a Mechanical Index between about 0.1 and about 5.0. In a further example, focused ultrasound is provided with a Mechanical Index between about 1.0 and about 5.0. In another example, the Mechanical Index is between about 1.5 and about 5.0. In certain aspects, the Mechanical Index is about 4.2 at a frequency of about 220 kHz. In other aspects, the Mechanical Index is about 3.5 at a frequency of about 500 kHz. In still other aspects, the Mechanical Index is about 2.9 at a frequency of about 700 kHz.

In one implementation, focused ultrasound treatment is provided with a pulse duration between 3.0 msec and 300 msec. In another implementation, the focused ultrasound treatment is provided with a pulse duration of 100 ms. In another implementations, the focused ultrasound treatment is provided at pulse duration of 150 ms.

In one example focused ultrasound treatment is provided with an acoustic pressure between about 0.55 MPa and about 2.5 MPa. In another example, focused ultrasound is delivered with an acoustic pressure of about 2 MPa. In another example, focused ultrasound is delivered with an acoustic pressure between 1.84 MPa and 1.9 MPa. In certain aspects, the acoustic pressure is greater than zero but less than about 2.5 MPa, such as about 0.55 MPa or about 1.9 MPa.

In one implementation, a session of focused ultrasound treatment lasts between three minutes and seven minutes. In another implementation, a session of focused ultrasound treatment lasts between five minutes and twenty minutes. In a further implementation, a session of focused ultrasound treatment lasts between three minutes and ten minutes. In a further implementation, a session of focused ultrasound treatment lasts between five minutes and twenty minutes. In a further implementation, a session of focused ultrasound treatment lasts between ten minutes and twenty minutes. In a further implementation, a session of focused ultrasound treatment lasts between ten minutes and thirty minutes. In a further implementation, a session of focused ultrasound treatment lasts between ten minutes and sixty minutes.

In one implementation, focused ultrasound treatment is provided with a carrier frequency between about 0.22 MHz and 3.0 MHz. In another implementation, focused ultrasound treatment is provided with a carrier frequency of about 220 kHz. In another implementation the focused ultrasound is delivered at about 500-700 kHz. In certain aspects, the carrier frequency is between about 0.22 and about 0.65 MHz. In certain aspects, the carrier frequency is about 0.6 MHz.

In one implementation, focused ultrasound treatment is provided with a spatial-peak temporal average intensity between 0.1 W/cm² and 8.5 W/cm². In another implementation, focused ultrasound treatment is provided with a spatial peak average intensity is between 3 W/cm² and 8.5 W/cm². In a further implementation the focused ultrasound treatment is provided with a spatial-peak temporal average intensity between about 1.33 W/cm² and about 5.64 W/cm².

In certain aspects, the I_{SPPA} can greater than zero and up to about 110 W/cm². In certain aspects, the I_{SPTA} can be greater than zero and up to about 8 W/cm². In certain aspects, the duty cycle can be greater than 0% and less about than 30%. In certain aspects, the duty cycle can be about 6.6%. In certain aspects, the duty cycle can be about 3.3%. In certain aspects, the duty cycle can be about 5%. In certain aspects, the pulse repetition rate can be between about 0.33 Hz and about 100 Hz. In certain aspects, the pulse repetition rate is about 0.33 Hz. In certain aspects, the pulse duration is between about 3.0 ms and about 300 ms. In certain aspects, the pulse duration is about 100 ms. In certain aspects, the acoustic pressure is between about 0.55 MPa and about 2.5 MPa. In certain aspects, the acoustic pressure is about 2 MPa. In certain aspects, the Mechanical Index is between about 1.5 and about 5.0. In certain aspects, the Mechanical Index is 4. In certain aspects, a session of focused ultrasound treatment lasts greater than zero minutes and up to about 30 minutes.

In certain aspects, the patient can be exposed to a cue associated with the tinnitus prior to or during delivering the ultrasound signal to the target site. Exposing the patient to a cue associated with the patient's tinnitus before or during delivering a neuromodulation signal pre-activates (“primes”/“triggers”) or activates the relevant neural circuits associated with tinnitus in individuals suffering from tinnitus creating a more receptive state for neuromodulatory input. Cue priming in neuromodulation can enhance the specificity and effectiveness of the intervention by aligning neuromodulatory stimuli with the brain's natural rhythms, neurocircuitry, or cognitive states. As such, cue priming can be important because of the selective activation of tinnitus-associated sub-circuits by the cues, making these neurocircuits sensitive and receptive to neuromodulation therapy. The cue can be an auditory, tactile, or visual cue, and/or a cue that elicits anxiety in the patient. The therapy could also include exposing the patient to sensory deprivation before or during delivering of the ultrasound signal to the target site. It should be noted that the cue can be an auditory signal, other type of sensory signal, a signal that elicits anxiety, stress, or other components resulting in/associated with the tinnitus. Also, the patient could be exposed to complete silence as this can be very problematic for people with tinnitus, so the patient could be exposed to complete deprivation of auditory, visual and other sensory inputs.

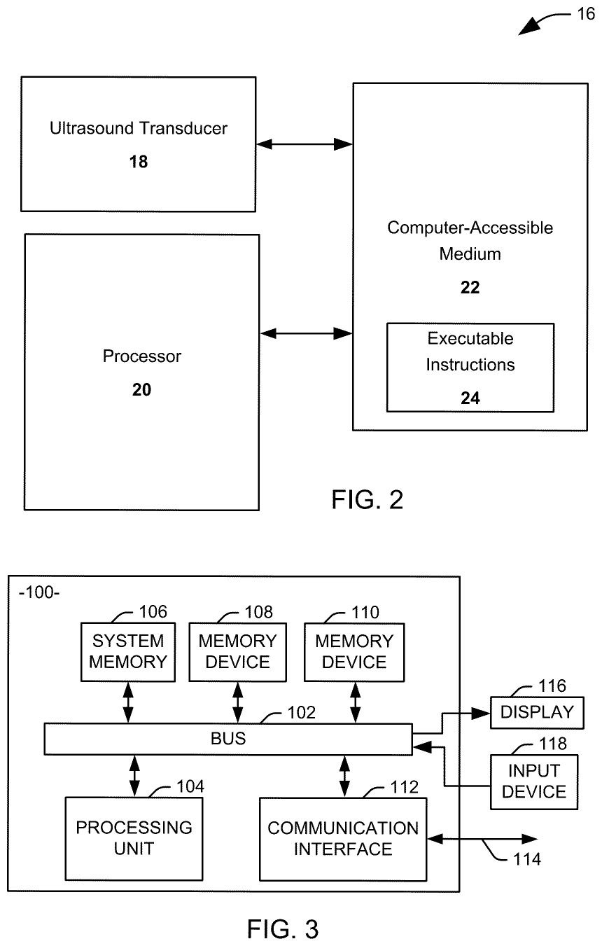

Referring to FIG. 2, in certain aspects a system of improving tinnitus is provided. System 16 can comprise ultrasound transducer 18, processor 20, and non-transitory computer-accessible medium 22 having stored thereon computer-executable instructions 22 which, when executed by processor 20, performs the following step: directs ultrasound transducer 18 to deliver an ultrasound signal to a target site of the patient's brain to improve tinnitus. The ultrasound signal can comprise a Mechanical Index of between about 1.5 and about 5.0 and/or an acoustic pressure of between about 0.55 MPa and about 2.5 MPa.

FIG. 3 is a schematic block diagram illustrating an exemplary system 100 of hardware components capable of implementing examples of the systems and methods as disclosed herein. System 100 can include various systems and subsystems. System 100 can be, for example, any of personal computer, a laptop computer, a workstation, a computer system, an appliance, an application-specific integrated circuit (ASIC), a server, a server blade center, or a server farm.

System 100 can includes system bus 102, processing unit or processor 104, system memory 106, memory devices 108 and 110, communication interface 112 (e.g., a network interface), communication link 114, display 116 (e.g., a video screen), and input device 118 (e.g., a keyboard and/or a mouse). System bus 102 can be in communication with processing unit 104 and system memory 106. Additional memory devices 108 and 110, such as a hard disk drive, server, stand-alone database, or other non-volatile memory, can also be in communication with system bus 102. System bus 102 interconnects processing unit 104, memory devices 106-110, the communication interface 112, display 116, and input device 118. In some examples, system bus 102 also interconnects an additional port (not shown), such as a universal serial bus (USB) port.

System 100 can be implemented in a computing cloud. In such a situation, features of system 100, such as processing unit 104, communication interface 112, and memory devices 108 and 110 could be representative of a single instance of hardware or multiple instances of hardware with applications executing across the multiple of instances (i.e., distributed) of hardware (e.g., computers, routers, memory, processors, or a combination thereof). Alternatively, system 100 could be implemented on a single dedicated server.

Processing unit 104 can be a computing device and can include an application-specific integrated circuit (ASIC). Processing unit 104 executes a set of instructions to implement the operations of examples disclosed herein. The processing unit can include a processing core.

Additional memory devices 106, 108, and 110 can store data, programs, instructions, database queries in text or compiled form, and any other information that can be needed to operate a computer. Memories 106, 108 and 110 can be implemented as computer-readable media (integrated or removable) such as, for example, a memory card, disk drive, compact disk (CD), or server accessible over a network. In certain examples, memories 106, 108 and 110 can comprise text, images, video, and/or audio, portions of which can be available in formats comprehensible to human beings.

Additionally or alternatively, system 100 can access an external data source or query source through communication interface 112, which can communicate with the system bus 102 and communication link 114.

In operation, system 100 can be used to implement one or more parts of a focused ultrasound neuromodulation system in accordance with the present invention. Processing unit 104 executes one or more computer executable instructions originating from system memory 106 and/or memory devices 108 and 110. It will be appreciated that a computer readable medium can include multiple computer readable media each operatively connected to the processing unit.

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software code can be stored in a memory. Memory can be implemented within the processor or external to the processor. As used herein the term “memory” refers to any type of long term, short term, and volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The terms “computer readable medium” and “machine readable medium” includes, but is not limited to portable or fixed storage devices, optical storage devices, wireless channels, and/or various other storage mediums capable of storing that contain or carry instruction(s) and/or data. It will be appreciated that a “computer readable medium” or “machine readable medium” can include multiple media each operatively connected to a processing unit.

Each of the disclosed aspects and embodiments of the present disclosure may be considered individually or in combination with other aspects, embodiments, and variations of the disclosure. Additionally, when describing a range, all points within that range are included in this disclosure. Further, unless otherwise specified, none of the steps of the methods of the present disclosure are confined to any particular order of performance.

What is claimed is:

1. A method of improving tinnitus in a patient in need thereof comprising:
delivering an ultrasound signal to a target site of the patient's brain comprising medial geniculate nucleus, a nucleus accumbens, a caudate nucleus, a pulvinar nucleus, an insula, a subcallosal anterior cingulate area, a cingulate cortex, an auditory cortex or combinations thereof without delivering microbubbles to open the blood brain barrier, the ultrasound signal comprising a Mechanical Index of between about 1.5 and about 5.0 and/or an acoustic pressure of between about 0.55 MPa and about 2.5 MPa; and
improving the patient's tinnitus.

2. The method of claim 1, wherein the target site is the medial geniculate nucleus.

3. The method of claim 1, wherein the target site is the nucleus accumbens.

4. The method of claim 1, wherein the target site is the caudate nucleus.

5. The method of claim 1, wherein the target site is the pulvinar nucleus.

6. The method of claim 1, wherein the target site is the insula.

7. The method of claim 1, wherein the target site is the subcallosal anterior cingulate area.

8. The method of claim 1, wherein the target site is the cingulate cortex.

9. The method claim 1, wherein the target site is the auditory cortex.

10. The method of claim 1, further comprising exposing the patient to a cue associated with the tinnitus prior to or during delivering the ultrasound signal to the target site.

11. The method of claim 10, wherein the cue is an auditory cue or a visual cue.

12. The method of claim 10, wherein the cue associated with tinnitus is a cue that elicits anxiety in the patient.

13. The method of claim 1, further comprising exposing the patient to sensory deprivation before or during delivering of the ultrasound signal to the target site.

14. A non-transitory computer-accessible medium having stored thereon computer-executable instructions which, when executed by a processor, performs the following steps:
directs an ultrasound transducer to deliver an ultrasound signal to a target site of the patient's brain to improve tinnitus, the ultrasound signal comprising a Mechanical Index of between about 1.5 and about 5.0 and/or an acoustic pressure of between about 0.55 MPa and about 2.5 MPa.

15. A system to improve tinnitus comprising:
an ultrasound transducer;
a processor; and
a non-transitory computer-accessible medium having stored thereon computer-executable instructions which, when executed by a processor, performs the following step:
directs an ultrasound transducer to deliver an ultrasound signal to a target site of the patient's brain to improve tinnitus, the ultrasound signal comprising a Mechanical Index of between about 1.5 and about 5.0 and/or an acoustic pressure of between about 0.55 MPa and about 2.5 MPa.
